(19) United States
(12) Patent Application Publication
(10) Pub. No.: US 2005/0159476 A1
(43) Pub. Date: Jul. 21, 2005

(54) Pharmaceutical composition for preventing or treating respiratory disease

(75) Inventors: John Efthimiou, Oxford (GB); Toshihiko Komori, Tokyo (JP); Mikio Sakai, Tokyo (JP); Osamu Cynshi, Shizuoka (JP); Yoshiaki Takashima, Shizuoka (JP); Yoshiki Kawabe, Shizuoka (JP)

Correspondence Address: BROWDY AND NEIMARK, P.L.L.C.;624 NINTH STREET, NW, SUITE 300, WASHINGTON, DC, 20001-5303, US

(73) Assignee: Chugai Seiyaku Kabushiki Kaisha, Kita-ku (JP)

(21) Appl. No.: 10/508,604

(22) PCT Filed: Mar. 28, 2003
(86) PCT No.: PCT/JP03/03983

(30) Foreign Application Priority Data
Mar. 29, 2002 (JP) 2002-095493

(51) Int. Cl. A61K 31/381; A61K 31/343

(52) U.S. Cl. 514/443; 514/469

(57) ABSTRACT

An object of the present invention is to provide a pharmaceutical composition and a method for preventing or treating a respiratory disease. The pharmaceutical composition comprises a compound represented by formula (1): or a pharmaceutically acceptable salt thereof as an active ingredient.

### FIELD OF THE INVENTION

The present invention relates to a pharmaceutical composition and a method for preventing or treating a respiratory disease. More specifically, it relates to a pharmaceutical composition and a method for preventing or treating a respiratory disease comprising a 2,3-dihydrobenzofuran derivative or a 2,3-dihydrobenzothiophene derivative as an active ingredient, as well as a use of a 2,3-dihydrobenzofuran derivative or a 2,3-dihydrobenzothiophene derivative for the preparation of such a pharmaceutical composition.

### PRIOR ART

In recent years, respiratory diseases such as chronic obstructive pulmonary disease and asthma induced by environmental pollutants such as automobile exhaust gas, industrial flue gas and cigarette smoke have spread year by year. Chemical substances contained in environmental pollutants represented by smoke are thought to be deeply involved in the onset/development of these diseases by coming into contact with the airway during respiration to injure airway cells. Among major factors of cell injury is oxidative stress by oxidants such as active oxygen or peroxides highly contained in cigarette smoke or the like (Rahman I, MacNee W, Role of oxidants/antioxidants in smoking-induced lung diseases. Free Radical Biol Med 21: 669-681, 1966; Gilmour M I, Daniels M, McCrillis R C, Winsett D, Selgrade M K, Air pollutant-enhanced respiratory disease in experimental animals. Environ Health Perspect 109 (suppl 4): 619-622, 2001).

Experiments also showed that cigarette smoke not only induces cell injury but also promotes the production of. cytokines such as IL-8, G-CSF and MCP-1 (Masubuchi T, Koyama S, Sato E, Takamizawa A, Kubo K, Sekiguchi M, Nagai S, Izumi T, Smoke extract stimulates lung epithelial cells to release neutrophil and monocyte chemotactic activity. Am J Pathol 153:1903, 1998; Mio T, Romberger D J, Thompson A B, Robbins R A, Heires A, Rennard S I, Cigarette smoke induces interleukin-8 release from human bronchial epithelial cells. Am J Respir Crit Care Med 155:1770, 1997) to aggravate inflammatory reactions. Cigarette smoke was also reported to induce oxidation of LDL (Vruwink K G, Gershwin M E, Sachet P, Halpern G, Davis P A, J Invest Allergol Clin Immunol 6:294, 1996; Yamaguchi Y, Matsuno S, Kagota S, Haginaka J, Kunitomo M, Oxidants in cigarette smoke extract modify low-density lipoprotein in the plasma and facilitate atherogenesis in the aorta of Watanabe heritable hyperlipidemic rabbits. Atherosclerosis 156:109, 2001).

Antioxidants such as vitamin E are expected to be effective for treating or preventing the diseases because these inflammatory reactions are thought to be a response to some oxidative stress (MacNee W, Oxidative stress and lung inflammation in airways disease. Eu J Pharmacol 429:195, 2001; Centanni S, Santus P, Marco F D, Fumagalli F, Zarini S, Sala A, The potential role of tocopherol in asthma and allergies. BioDrugs 15:81, 2001). In fact, vitamin E was reported to inhibit cell injury caused by oxidative stress in alveolar epithelial cells, and also reported to inhibit the production of inflammatory cytokines such as IL-8 (Wu D, Koga T, Martin K R, Meydani M, Atherosclerosis 147:297, 1999).

However, vitamin E was reported to be clinically ineffective for chronic obstructive pulmonary disease (Rautalahti M, Virtamo J, Haukka J, Heinonen O P, Sundvall J, Albanes D, Huttunen J K, The effect of alpha-tocopherol and beta-carotene supplementation on COPD symptoms. Am J Respir Crit Care Med 156:1447, 1997).

This is probably because vitamin E acts as an antioxidant but sometimes also acts as an oxidation promoter in some conditions. This is supported by the report that a large amount of vitamin E administered remains unconsumed though oxidation proceeds in the lesion of arteriosclerosis in which oxidative stress seems to have an important role (Suarna C, Dean R T, May J, Stocker R, Human atherosclerotic plaque contains both oxidized lipids and relatively large amounts of a-tocopherol and ascorbate. Arterioscler Thromb Vasc Biol 15:1616, 1995). Moreover, a long-term pretreatment is required for vitamin E to produce cell injury inhibitory effect (for example, a 20 hr-pretreatment was conducted in Wu D, Koga T., Martin K R, Meydani M, Atherosclerosis 147:297, 1999). Thus, vitamin E may be affected by biological oxidative stress or metabolism before reaching the lesion on which the vitamin acts, thereby failing to give a sufficient effect to improve symptoms of a respiratory disease.

In this way, vitamin E is not sufficiently effective as an agent for preventing or treating a respiratory disease, and therefore, it would be desirable to develop an alternative agent for preventing or treating a respiratory disease.

### SUMMARY OF THE INVENTION

The present invention provides a pharmaceutical composition and a method for preventing or treating a respiratory disease.

As a result of extensive research to solve the above problems, we found that a compound represented by formula (1) has an excellent prophylactic or therapeutic effect for a respiratory disease.

Accordingly, the present invention provides a pharmaceutical composition for preventing or treating a respiratory disease comprising a compound represented by formula (1): 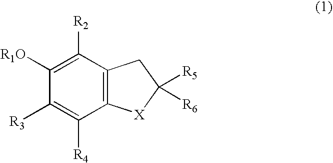
wherein
X represents an oxygen atom or a sulfur atom;
R₁ represents a hydrogen atom or an acyl or arylalkyloxycarbonyl group;
R₂, R₃ and R₄ are identical or different and each represents a hydrogen atom or a lower alkyl group; and
R₅ and R₆ are identical or different and each represents a hydrogen atom, a carboxyl group or an optionally substituted alkyl group; or
R₅ and R₆ may be combined to form a cycloalkyl group or a saturated heterocyclic group;
   or a pharmaceutically acceptable salt thereof as an active ingredient.

The present invention also provides a method for preventing or treating a respiratory disease comprising administering a prophylactically or therapeutically effective amount of a compound represented by formula (1) or a pharmaceutically acceptable salt thereof, to a patient in need of such prevention or treatment.

The present invention also provides a use of a compound represented by formula (1) or a pharmaceutically acceptable salt thereof for the preparation of a pharmaceutical composition for preventing or treating a respiratory disease.

### BRIEF DESCRIPTION OF THE DRAWING

FIG. 1 is a graph showing the protective effect of a compound of the present invention against cell injury induced by oxidized LDL in human A549 cells.

FIG. 2 is a graph showing the protective effect of a compound of the present invention against cell injury induced by t-butyl hydroperoxide (T-BuOOH) in human A549 cells.

### DETAILED DESCRIPTION OF THE INVENTION

The compounds represented by formula (1) of the present invention are known. JP 6-206842A/1994 and WO94/08930 describe that these compounds have an antioxidant effect. JP 10-72458A/1998 and WO97/49388 describe that these compounds in vitro inhibit injury caused by oxidized LDL in renal cells. However, it has not been known that compounds of formula (1) of the present invention are effective for preventing or treating a respiratory disease.

The compounds represented by formula (1) used in the present invention can be synthesized by any of the processes described in:
JP 6-206842A/1994 and corresponding U.S. Pat. No. 5,574,178 or EP 0665208B;
JP 7-330759A/1995 and corresponding U.S. Pat. No. 5,789,436 or EP 0791589A;
JP 10-72458A/1998 and corresponding U.S. Pat. No. 6,133,279 or EP 0950405A; or
JP 11-35568A/1999 and corresponding EP 0995437A.

In formula (1) of the present invention, X preferably represents an oxygen atom.

Examples of the acyl group represented by R₁ include aliphatic acyl groups containing 1-10 carbon atoms and aromatic acyl groups containing 7-10 carbon atoms. Preferred examples of the aliphatic acyl groups include formyl, acetyl, propionyl and hexanoyl groups, and specific examples of the aromatic acyl groups include benzoyl group. Aliphatic acyl groups are preferred, among which aliphatic acyl groups containing 1-6 carbon atoms are more preferred and acetyl is especially preferred.

Examples of the arylalkyloxycarbonyl group represented by R₁ preferably include those containing 7-12 carbon atoms. Preferred examples include benzyloxycarbonyl and naphthylmethoxycarbonyl groups.

Preferably, R₁ is a hydrogen atom or an acyl group, more preferably a hydrogen atom and an acetyl group, especially a hydrogen atom.

The lower alkyl group represented by R₂ and R₃ is preferably a straight or branched alkyl group containing 1-6 carbon atoms, more preferably a branched alkyl group containing 3-4 carbon atoms, especially t-butyl.

R₄ preferably represents a hydrogen atom or an alkyl group containing 1-4 carbon atoms, more preferably a hydrogen atom or a branched alkyl group containing 3-4 carbon atoms, especially a hydrogen atom.

Examples of the alkyl group represented by R₅ and R₆ preferably include straight or branched alkyl groups containing 1-10 carbon atoms. Specific examples include methyl, ethyl, n-propyl, isopropyl, n-butyl, isobutyl, s-butyl, n-pentyl, isopentyl, sec-pentyl, t-pentyl, neopentyl, n-hexyl, isohexyl, ethylbutyl, n-heptyl, isoheptyl, ethylpentyl, n-octyl, ethylhexyl, propylpentyl, nonyl and decyl groups. More preferred alkyl groups represented by R₅ and R₆ are straight or branched alkyl groups containing 3-8 carbon atoms.

Examples of the cycloalkyl group formed by R₅ and R₆ preferably include cycloalkyl groups containing 3-8 carbon atoms. Specific examples include cyclopropyl, cyclobutyl, cyclopentyl and cyclooctyl groups, more preferably cycloalkyl groups containing 5-8 carbon atoms, especially cycloalkyl groups containing 4-7 carbon atoms.

Examples of the saturated heterocyclic group formed by R₅ and R₆ preferably include saturated 5-12-membered heterocyclic groups containing 1-3 oxygen or sulfur atoms. Specific examples include tetrahydrofuran, tetrahydrothiophene and tetrahydropyranyl groups. More preferred are saturated 5-6-membered heterocyclic groups containing one oxygen atom or sulfur atom, and especially preferred are saturated 6-membered heterocyclic groups containing one oxygen or sulfur atom.

Examples of the substituent for the optionally substituted alkyl groups represented by R₅ and R₆ include, for example, halogen, lower alkyl, lower alkenyl, lower alkynyl, hydroxyl, amino, mono- or di-alkylamino, carboxyl, acyl, cyano, alkoxy, aryloxy, nitro, halogenoalkyl, aryl and heteroaryl groups, preferably aryl, amino and mono- or di-alkylamino groups, more preferably aryl and amino groups. The lower alkyl group here means a straight or branched alkyl group containing 1-6 carbon atoms.

Especially preferred groups for R₅ and R₆ are straight alkyl groups containing 4-7 carbon atoms, specifically n-butyl, n-pentyl, n-hexyl or n-heptyl group.

The pharmaceutically acceptable salt of the compound represented by formula (1) of the present invention can be formed when the compound of formula (1) has a group capable of forming an addition salt with an acid or a base in R₅ or R₆. Examples of the acid used for forming acid addition salt include, for example, inorganic acids such as hydrochloric acid, hydrobromic acid, nitric acid, sulfuric acid and phosphoric acid; and organic acids such as acetic acid, lactic acid, oxalic acid, glycolic acid, tartaric acid, malic acid and citric acid. Examples of the base used for forming base addition salt include bases such as methylamine, ethylamine, ethanolamine, pyridine, piperidine, morpholine and triethylamine.

Preferred specific examples of the compound represented by formula (1) of the present invention are as follows:
5-acetoxy-4,6-di-t-butyl-2,2-dimethyl-2,3-dihydrobenzofuran;
5-acetoxy-4,6-di-t-butyl-2,2-diethyl-2,3-dihydrobenzofuran;
5-acetoxy-4,6-di-t-butyl-2,2-dimethyl-7-propyl-2,3-dihydrobenzofuran;
5-acetoxy-2,2-di-i-amyl-4,6-di-t-butyl-2,3-dihydrobenzofuran;
5-acetoxy-4,6-di-t-butyl-2-(5-hydroxy-4-methyl-3(E)-pentenyl)-2-methyl-2,3-dihydrobenzofuran;
5-acetoxy-4,6-di-t-butyl-2-hydroxymethyl-2-methyl-2,3-dihydrobenzofuran;
4,6-di-t-butyl-5-hydroxy-2,3-dihydrobenzofuran;
4,6-di-t-butyl-5-hydroxy-2,2-dimethyl-2,3-dihydrobenzofuran;
4,6-di-t-butyl-5-hydroxy-2-methyl-2,3-dihydrobenzofuran;
4,6-di-t-butyl -2,2-diethyl-5-hydroxy-2,3-dihydrobenzofuran;
4,6-di-t-butyl-2,2-di-n-propyl-5-hydroxy-2,3-dihydrobenzofuran;
4,6-di-t-butyl-2,2-di-i-propyl-5-hydroxy-2,3-dihydrobenzofuran;
4,6-di-t-butyl-2,2-di-n-butyl-5-hydroxy-2,3-dihydrobenzofuran;
4,6-di-t-butyl-2,2-di-n-pentyl-5-hydroxy-2,3-dihydrobenzofuran;
2,2-di-i-amyl-4,6-di-t-butyl-5-hydroxy-2,3-dihydrobenzofuran;

14,6-di-t-butyl-2,2-di-n-hexyl-5-hydroxy-2,3-dihydrobenzofuran;
4,6-di-t-butyl-2,2-di-n-heptyl-5-hydroxy-2,3-dihydrobenzofuran;
4,6-di-t-butyl-2,2-di-n-octyl-5-hydroxy-2,3-dihydrobenzofuran;
4,6-di-t-butyl-5-hydroxy-2-octyl-2,3-dihydrobenzofuran;
2,4,6-tri-t-butyl-5-hydroxy-2,3-dihydrobenzofuran;
4,6-di-t-butyl-2,2-dibenzyl-5-hydroxy-2,3-dihydrobenzofuran;
4,6-di-t-butyl-2-chloromethyl-5-hydroxy-2,3-dihydrobenzofuran;
5-hydroxy-4,6-di-t-butyl-2,2-dimethyl-7-propyl-2,3-dihydrobenzofuran;
4,6-di-t-butyl-5-hydroxy-2-(5-hydroxy-4-methyl-3(E)-pentenyl)-2-methyl-2,3-dihydrobenzofuran;
4,6-di-t-butyl-5-hydroxy-2-hydroxymethyl-2-methyl-2,3-dihydrobenzofuran;
2-aminomethyl-4,6-di-t-butyl-5-hydroxy-2-methyl-2,3-dihydrobenzofuran;
4,6-di-t-butyl-5-hydroxy-2-methyl-2,3-dihydrobenzofuran-2-carboxylic acid;
4,6-di-t-butyl-5-hydroxy-2,3-dihydrobenzofuran-2-spiro-1′-cycloheptane;
4,6-di-t-butyl-5-hydroxy-2,3-dihydrobenzofuran-2-spiro-1′-cyclooctane;
4,6-di-t-butyl-5-hydroxy-2,3-dihydrobenzofuran-2-spiro-4′-tetrahydropyran;
4,6-di-t-butyl -5-hydroxy-2,3-dihydrobenzofuran-2-spiro-1′-cyclopentane;
4,6-di-t-butyl-5-hydroxy-2,3-dihydrobenzofuran-2-spiro-1′-cyclohexane;
4,6-di-t-butyl-5-hydroxy-2,3-dihydrobenzofuran-2-spiro-4′-tetrahydrothiopyran;
5-hydroxy-2,2,4,6-tetramethyl-2,3-dihydrobenzofuran;
4,7-di-t-butyl-5-hydroxy-2,2-dimethyl-2,3-dihydrobenzofuran;
4,6-dimethyl-2,2-di-n-pentyl-5-hydroxy-2,3-dihydrobenzofuran;
6-t-butyl-5-hydroxy-2,3-dihydrobenzofuran-2-spiro-11-cycloheptane;
5-acetoxy-4,6-di-t-butyl-2,2-di-n-pentyl-2,3-dihydrobenzothiophene;
5-acetoxy-4,6-di-t-butyl-2-iodomethyl-2-methyl-2,3-dihydrobenzothiophene;
5-acetoxy-4,6-di-t-butyl-2-(N,N-dimethylaminomethyl)-2-methyl-2,3-dihydrobenzothiophene;
5-acetoxy-2-acetoxymethyl-4,6-di-t-butyl-2-methyl-2,3-dihydrobenzothiophene;
4,6-di-t-butyl-5-hydroxy-2,2-di-n-pentyl-2,3-dihydrobenzothiophene;
4,6-di-t-butyl-5-hydroxy-2-methyl-2,3-dihydrobenzothiophene;
4,6-di-t-butyl-5-hydroxy-2,2-dimethyl-2,3-dihydrobenzothiophene;
4,6-di-t-butyl-5-hydroxy-2,3-dihydrobenzothiophene;
4,6-di-t-butyl-5-hydroxy-2-(N,N-dimethylaminomethyl)-2-methyl-2,3-dihydrobenzothiophene;
4,6-di-t-butyl-5-hydroxy-2-hydroxymethyl-2-methyl-2,3-dihydrobenzothiophene;
4,6-di-t-butyl-5-hydroxy-2-methyl-2-(4,8-dimethylnona-3(E),7-dienyl)-2,3-dihydrobenzothiophene;
4,6-di-t-butyl-5-hydroxy-2-methyl-2-(4,8-dimethylnonyl)-2,3-dihydrobenzothiophene;
4,6-di-t-butyl-5-hydroxy-2,3-dihydrobenzothiophene-2-spiro-1′-cyclohexane; and
4,6-di-t-butyl-5-hydroxy-2,3-dihydrobenzothiophene-2-spiro-4′-tetrahydropyran.

The compound represented by formula (1) of the present invention is useful for preventing or treating a respiratory disease because it shows excellent injury inhibitory effect on bronchial epithelial cells and alveolar epithelial cells that constitute the airway and directly come into contact with expiratory air.

The respiratory diseases include, for example, respiratory tract diseases.

The respiratory tract diseases include, for example, bronchial asthma, pulmonary emphysema, chronic obstructive pulmonary emphysema, centrilobular emphysema, panacinar pulmonary emphysema, acute bronchitis, chronic bronchitis, chronic obstructive bronchitis, reactive respiratory tract diseases, cystic fibrosis, bronchiectasis, acquired bronchiectasis, Kartagener's syndrome, apneumatosis, acute apneumatosis, chronic apneumatosis, pneumonia, essential thrombocytopenia, legionellosis, parrot disease, fibroplastic pneumoconiocis, diseases caused by organic dust, diseases caused by irritant gas and chemical substances, pulmonary hypersensitivity, chronic obstructive pulmonary disease and idiopathic invasive lung disorder, among which the diseases preferably treated by the pharmaceutical composition of the present invention are pulmonary emphysema, chronic bronchitis, chronic obstructive pulmonary disease, asthma, pneumonia and bronchitis, more preferably chronic obstructive pulmonary disease and asthma.

The pharmaceutical composition of the present invention can be formulated into various dosage forms by combining a compound represented by formula (1) as an active ingredient with a physiologically acceptable solid or liquid carrier depending on the administration route. Suitable dosage forms include, for example, formulations for topical, oral, buccal, intranasal, parenteral (for example, intravenous, intramuscular or subcutaneous) or rectal administration or for inhalation or insufflation, specifically tablets, pills, capsules, granules, solutions, syrups, suspensions, emulsions, injections and aerosols.

The dose of the compound represented by formula (1) of the present invention is appropriately determined depending on the age of the patient, the severity of the condition, the route of administration, etc., for example in the range of 0.1-1000 mg, preferably 10-500 mg daily per adult. This dose may be administered once or divided into several portions.

The following examples further illustrate the present invention, but it should be understood that the invention is not limited to these examples.

### EXAMPLES

The following compounds were used in the following tests.
Compound 1: 4,6-di-t-butyl-5-hydroxy-2,3-dihydrobenzofuran;
Compound 2: 4,6-di-t-butyl-5-hydroxy-2,2-dimethyl-2,3-dihydrobenzofuran;
Compound 3: 4,6-di-t-butyl-2,2-di-n-pentyl-5-hydroxy-2,3-dihydrobenzofuran;
Compound 4: 4,6-di-t-butyl-2,2-dibenzyl-5-hydroxy-2,3-dihydrobenzofuran;
Compound 5: 2-aminomethyl-4,6-di-t-butyl-5-hydroxy-2-methyl-2,3-dihydrobenzofuran;
Compound 6: 4,6-di-t-butyl-5-hydroxy-2-methyl-2,3-dihydrobenzofuran-2-carboxylic acid;
Compound 7: 4,6-di-t-butyl-5-hydroxy-2,3-dihydrobenzofuran-2-spiro-1′-cycloheptane;
Compound 8: 4,6-di-t-butyl-5-hydroxy-2,3-dihydrobenzofuran-2-spiro-4′-tetrahydropyran; ‘Compound 9: 4,6-di-t-butyl-5-hydroxy-2,3-dihydrobenzofuran-2-spiro-1′-cyclopentane;
Compound 10: 4,6-di-t-butyl-5-hydroxy-2,3-dihydrobenzofuran-2-spiro-1′-cyclohexane;
Compound 11: 4,6-di-t-butyl-5-hydroxy-2,3-dihydrobenzofuran-2-spiro-4′-tetrahydrothiopyran;
Compound 12: 4,6-di-t-butyl-5-hydroxy-2,2-dimethyl-2,3-dihydrobenzothiophene;
Compound 13: 4,6-di-t-butyl-5-hydroxy-2,3-dihydrobenzothiophene-2-spiro-1′-cyclohexane;
Compound 14: 4,6-di-t-butyl-5-hydroxy-2,3-dihydrobenzothiophene-2-spiro-4′-tetrahydropyran;
Compound 15: 5-hydroxy-2,2,4,6-tetramethyl-2,3-dihydrobenzofuran;
Compound 16: 4,7-di-t-butyl-5-hydroxy-2,2-dimethyl-2,3-dihydrobenzofuran;
Compound 17: 4,6-dimethyl-2,2-di-n-pentyl-5-hydroxy-2,3-dihydrobenzofuran; and
Compound 18: 6-t-butyl-5-hydroxy-2,3-dihydrobenzofuran-2-spiro-1′-cycloheptane.

The structures of these test compounds are shown in Table 1.

TABLE 1
Structures of test compounds

| | |
|---|---|
| | (1) |
| 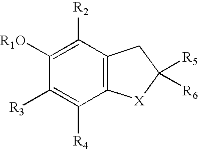 | |

| Compound | X | R1 | R2 | R3 | R4 | R5 | R6 |
|---|---|---|---|---|---|---|---|
| 1 | O | H | t-Bu | t-Bu | H | H | H |
| 2 | O | H | t-Bu | t-Bu | H | Me | Me |
| 3 | O | H | t-Bu | t-Bu | H | n-pentyl | n-pentyl |
| 4 | O | H | t-Bu | t-Bu | H | Benzyl | benzyl |
| 5 | O | H | t-Bu | t-Bu | H | Me | CH₂NH₂ |
| 6 | O | H | t-Bu | t-Bu | H | Me | COOH |
| 7 | O | H | t-Bu | t-Bu | H | 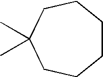 | |
| 8 | O | H | t-Bu | t-Bu | H | 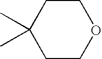 | |
| 9 | O | H | t-Bu | t-Bu | H | 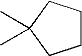 | |
| 10 | O | H | t-Bu | t-Bu | H | 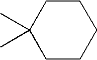 | |
| 11 | O | H | t-Bu | t-Bu | H | 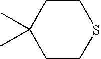 | |
| 12 | S | H | t-Bu | t-Bu | H | Me | Me |
| 13 | S | H | t-Bu | t-Bu | H | 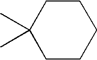 | |
| 14 | S | H | t-Bu | t-Bu | H | 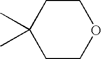 | |
| 15 | O | H | Me | Me | H | Me | Me |
| 16 | O | H | t-Bu | H | t-Bu | Me | Me |
| 17 | O | H | Me | Me | H | n-pentyl | n-pentyl |

| | | | | | | |
|---|---|---|---|---|---|---|
| 18 | O | H | H | t-Bu | H | 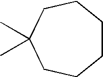 |

### Test Example 1

### Protective Effect Against Cell Injury Induced by Oxidized Low-Density Lipoprotein (Oxidized LDL) in Human A549 Cells (1)

Oxidized LDL was prepared by maintaining 1 mg/mL of rabbit LDL at 37° C for 24 hours in PBS (-) containing 10 μmol/L CUSO₄.

Human A549 cells used in this test are available under accession number ATCC-CCL-185 and have the properties of type II alveolar epithelial cells (Lieber M, Smith B, Szakal A, Nelson-Rees W, Todaro G., A continuous tumor-cell line from a human lung carcinoma with properties of type II alveolar epithelial cells. Int J Cancer Jan. 15, 1976; 17(1):62-70).

These cells were dispersed in F12K medium containing 10% FBS and plated on a 24-well plate at 2.5×10⁴ cells/500 μL/well. These cells were incubated at 37° C. for 18 hours and then the medium was replaced by F12K medium containing 2% FBS. At this time, 2.5 μL of a solution or a suspension of each test compound in ethanol was added at the concentrations of the test compound of 3 μmol/L and 30 μmol/L in each well.

After incubation at 37° C. for 6 hours, 50 μL of a suspension of oxidized LDL was added per well at the concentration of oxidized LDL of 100 μg/mL in each well.

After incubation at 37° C. for 24 hours, 200 μL of the supernatant was collected from each well and the amount of lactate dehydrogenase (LDH) leaked from cells was determined by the SFBC method.

Further test was carried out in the same manner as the above test with the exception that each test compound was added to the cell culture after incubation for 6 hours following medium replacement and immediately before oxidized LDL was added thereto rather than when the medium was replaced with F12K medium containing 2% FBS.

Higher LDH levels in the supernatant indicate that greater cell injury is induced by oxidized LDL. Separately, one experiment was carried out using a well in the same manner as the above test with the exception that no test compound was added to the well. The level of LDH in this well was defined as 0% cell protection. In addition, other experiment was carried out using another well without adding either test compound or oxidized LDL but with adding saline instead. The level of LDH in this well was defined as 100% cell protection. These levels of LDH were used for calculating cell protection of each test compound used in the above test. The results are shown in Table 2.

TABLE 2
Protective effect of compounds of the present
invention against cell injury in A549 cells

| | % Cell protection in the | | % Cell protection in the | |
|---|---|---|---|---|
| | case where oxidized LDL | | case where oxidized LDL | |
| | was added 6 hrs after | | was added immediately after | |
| | adding test compound | | adding test compound | |
| Comp. | 3 μmol/L | 30 μmol/L | 3 μmol/L | 30 μmol/L |
| 1 | 11.3 ± 1.6 | 99.6 ± 0.9 | 40.4 ± 8.3 | 98.8 ± 0.2 |
| 2 | 0.5 ± 1.3 | 99.5 ± 1.1 | 16.9 ± 10.5 | 100.1 ± 1.5 |
| 3 | 18.7 ± 2.1 | 95.0 ± 2.5 | 14.3 ± 2.3 | 85.4 ± 1.6 |
| 4 | 6.1 ± 2.5 | 94.1 ± 0.4 | 5.3 ± 2.5 | 95.3 ± 3.4 |
| 5 | 6.4 ± 0.8 | 96.5 ± 0.5 | 6.9 ± 3.1 | 97.1 ± 0.8 |
| 6 | 8.5 ± 0.8 | 65.3 ± 2.9 | 9.1 ± 1.2 | 46.7 ± 2.9 |
| 7 | 1.6 ± 6.0 | 98.2 ± 2.1 | −0.8 ± 2.8 | 98.4 ± 1.1 |
| 8 | 10.6 ± 6.2 | 95.0 ± 5.3 | 10.8 ± 5.2 | 93.6 ± 2.9 |
| 9 | −1.6 ± 4.4 | 98.5 ± 1.8 | −1.5 ± 4.1 | 99.2 ± 2.2 |
| 10 | −0.7 ± 1.5 | 97.8 ± 2.1 | 0.9 ± 1.9 | 96.9 ± 4.8 |
| 11 | 3.5 ± 2.6 | 99.3 ± 1.1 | 4.6 ± 2.6 | 98.5 ± 0.8 |
| 12 | 9.9 ± 6.8 | 98.7 ± 2.9 | 11.1 ± 7.4 | 96.6 ± 2.0 |
| 13 | 10.8 ± 3.9 | 98.0 ± 1.0 | 11.3 ± 3.5 | 97.1 ± 4.3 |
| 14 | 7.0 ± 3.5 | 96.1 ± 1.2 | 3.2 ± 1.3 | 98.9 ± 2.4 |
| 15 | 30.0 ± 10.2 | 100.6 ± 0.9 | 36.1 ± 14.1 | 99.6 ± 0.7 |
| 16 | 100.3 ± 1.0 | — | 99.1 ± 0.9 | 98.4 ± 0.6 |
| 17 | 97.8 ± 1.4 | — | 97.7 ± 1.1 | 98.1 ± 3.6 |
| 18 | 98.7 ± 1.3 | — | 96.2 ± 0.4 | 97.4 ± 1.0 |

Each value means average ± standard error.

As can be seen from Table 2, the compounds of the sent invention inhibit cell injury in human A549 cells.

### Test Example 2

### Protective Effect Against Cell Injury Induced by Oxidized LDL in Human A549 Cells (22)

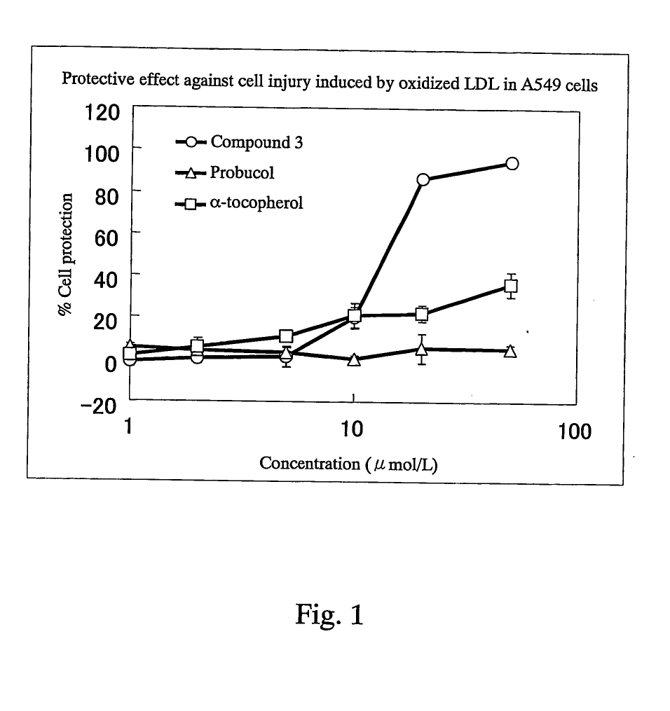

This test example was carried out in a similar manner to test example 1 except that Compound 3, probucol and α-tocopherol were used as test compounds. Probucol and α-tocopherol were tested for comparison with the compounds of the present invention. In this test, the test compounds were added to the cell culture after incubation for 6 hours following medium replacement and immediately before oxidized LDL was added. The results are shown in FIG. 1.

As can be seen from FIG. 1, in the case where Compound 3 is used, % cell protection begins to sharply increase when the concentration of Compound 3 exceeds about 10 μmol/L while, in the case where probucol or α-tocopherol is used, it does not increase or only slowly increases.

### Test Example 3

### Protective Against Cell Injury Induced by Oxidized LDL in Normal Human Bronchial/Tracheal Epithelial Cells

This test example was carried out in a similar manner to test example 1 except that human A549 cells were replaced with normal human bronchial/tracheal epithelial cells (NHBE w/RA: BioWhittaker Co.), cell culture medium was changed from F12K medium containing 10% FBS to BEGM® medium, and the replacement of medium was not made. The results are shown in Table 3.

TABLE 3
Protective effect of compounds of the present invention against cell
injury in normal human bronchial/tracheal epithelial cells

| | | | % Cell protection in the | |
|---|---|---|---|---|
| | % Cell protection in the | | case where oxidized LDL | |
| | case where oxidized LDL | | was added immediately | |
| | was added 6 hrs after | | after adding test | |
| | adding test compound | | compound | |
| Comp. | 3 μmol/L | 30 μmol/L | 3 μmol/L | 30 μmol/L |
| 1 | 5.9 ± 4.7 | 74.7 ± 1.4 | 35.6 ± 16.8 | 59.2 ± 7.6 |
| 2 | 32.8 ± 2.9 | 27.7 ± 2.7 | 71.1 ± 1.3 | 76.1 ± 3.4 |
| 3 | 49.8 ± 6.4 | 70.8 ± 2.0 | 9.8 ± 3.3 | 66.2 ± 2.6 |
| 4 | 12.3 ± 4.3 | 47.8 ± 1.6 | 9.1 ± 2.3 | 53.5 ± 3.1 |
| 5 | 6.3 ± 4.5 | 33.6 ± 1.8 | 0.8 ± 4.6 | 48.3 ± 8.1 |
| 6 | −0.9 ± 1.8 | 44.8 ± 3.2 | 0.3 ± 2.0 | 30.3 ± 3.1 |
| 7 | 7.7 ± 3.6 | 58.7 ± 5.1 | 11.3 ± 1.1 | 73.4 ± 0.4 |
| 8 | 10.1 ± 6.0 | 74.3 ± 1.5 | 13.2 ± 1.4 | 68.9 ± 0.8 |
| 9 | 3.7 ± 6.7 | 18.1 ± 2.3 | 6.1 ± 1.1 | 71.1 ± 2.3 |
| 10 | 5.3 ± 2.6 | 37.3 ± 4.3 | 13.1 ± 6.4 | 57.0 ± 2.5 |
| 11 | 3.0 ± 3.2 | 52.0 ± 2.2 | 7.5 ± 7.6 | 40.9 ± 5.3 |
| 12 | 10.9 ± 2.1 | 21.2 ± 2.0 | 10.9 ± 3.8 | 48.7 ± 1.2 |
| 13 | −3.4 ± 7.1 | 37.8 ± 0.7 | −4.8 ± 2.9 | 49.2 ± 1.7 |
| 14 | −0.5 ± 1.9 | 63.3 ± 0.8 | −1.0 ± 5.3 | 60.6 ± 4.0 |
| 15 | 45.9 ± 4.5 | 79.5 ± 6.4 | 40.8 ± 3.3 | 70.6 ± 4.9 |
| 16 | 54.9 ± 3.4 | — | 62.6 ± 1.6 | 42.0 ± 2.2 |
| 17 | 44.8 ± 5.9 | — | 51.0 ± 5.0 | 57.6 ± 0.6 |
| 18 | 55.7 ± 3.1 | — | 46.4 ± 1.4 | 35.5 ± 1.9 |

Each value means average ± standard error.

As can be seen from Table 3, the compounds of the present invention inhibit cell injury in human bronchial/tracheal epithelial cells.

### Test Example 4

### Protective Effect Against Cell Injury Induced by t-butyl Hydroperoxide in Human A549 Cells

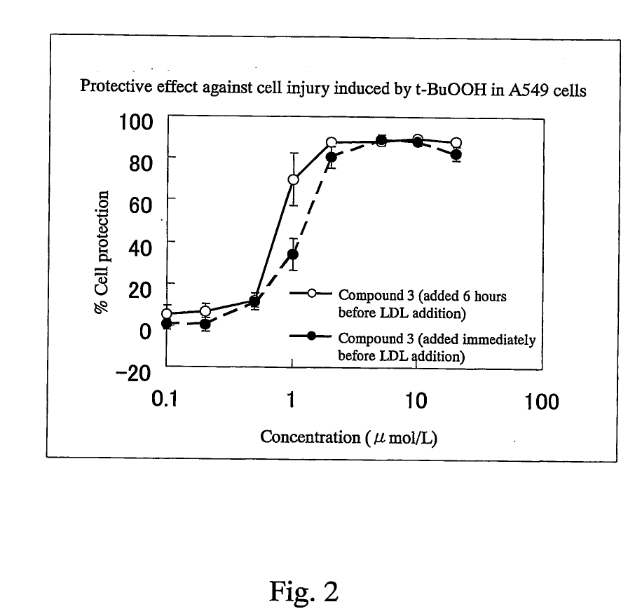

This test example was carried out in a similar manner to test example 1 except that only Compound 3 was used as a test compound and that a solution of t-butyl hydroperoxide (t-BuOOH) in saline was added at 200 μmol/mL in each well instead of a suspension of oxidized LDL at 100 μg/mL in each well. The results are shown in FIG. 2.

As can be seen from FIG. 2, Compound 3 inhibits cell injury induced by a peroxide, t-butyl hydroperoxide in human A549 cells.

### ADVANTAGES OF THE INVENTION

The compounds represented by formula (1) of the present invention have a protective effect for respiratory cells and are useful for preventing or treating a respiratory disease.

1-18. (canceled)

19. A method for preventing or treating a respiratory disease comprising administering to a patient in need of such prevention or treatment a prophylactically or therapeutically effective amount of a compound represented by formula (1): 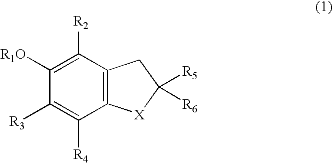
wherein
X represents an oxygen atom or a sulfur atom;
R₁ represents a hydrogen atom or an acyl or arylalkyloxycarbonyl group;
R₂, R₃ and R₄ are identical or different and each represents a hydrogen atom or a lower alkyl group; and
R₅ and R₆ are identical or different and each represents a hydrogen atom, a carboxyl group or an optionally substituted alkyl group; or
R₅ and R₆ may be combined to form a cycloalkyl group or a saturated heterocyclic group;
or a pharmaceutically acceptable salt thereof.

20. The method of claim 19 comprising administering a compound represented by formula (1) to the lung or digestive tract.

21. The method of claim 19 wherein X represents an oxygen atom.

22. The method of claim 19 wherein R₁ represents a hydrogen atom.

23. The method of claim 19 wherein R₂ and R₃ each represents a lower alkyl group.

24. The method of claim 23 wherein the lower alkyl group is t-butyl group.

25. The method of claim 19 wherein R₄ represents a hydrogen atom.

26. The method of claim 19 wherein R₅ and R₆ each represents an alkyl group.

27. The method of claim 26 wherein the alkyl group is n-butyl, n-pentyl, n-hexyl or n-heptyl group.

28. The method of claim 19 wherein the compound represented by formula (1) is 4,6-di-t-butyl-5-hydroxy-2,2-di-n-butyl-2,3-dihydrobenzofuran, 4,6-di-t-butyl-5-hydroxy-2,2-di-n-pentyl-2,3-dihydrobenzofuran, 4,6-di-t-butyl-5-hydroxy-2,2-di-n-hexyl-2,3-dihydrobenzofuran or 4,6-di-t-butyl-5-hydroxy-2,2-di-n-heptyl-2,3-dihydrobenzofuran.

29. The method of claim 28 wherein the compound represented by formula (1) is 4,6-di-t-butyl-5-hydroxy-2,2-di-n-pentyl-2,3-dihydrobenzofuran.

30. The method of claim 19 wherein the respiratory disease is a respiratory tract disease.

31. The method of claim 30 wherein the respiratory tract disease is selected from the group consisting of bronchial asthma, pulmonary emphysema, chronic obstructive pulmonary emphysema, centrilobular emphysema, panacinar pulmonary emphysema, acute bronchitis, chronic bronchitis, chronic obstructive bronchitis, reactive respiratory tract diseases, cystic fibrosis, bronchiectasis, acquired bronchiectasis, Kartagener's syndrome, apneumatosis, acute apneumatosis, chronic apneumatosis, pneumonia, essential thrombocytopenia, legionellosis, parrot disease, fibroplastic pneumoconiocis, diseases caused by organic dust, diseases caused by irritant gas and chemical substances, pulmonary hypersensitivity, chronic obstructive pulmonary disease and idiopathic invasive lung disorder.

32. The method of claim 31 wherein the respiratory tract disease is pulmonary emphysema.

33. The method of claim 31 wherein the respiratory tract disease is acute bronchitis, chronic bronchitis or chronic obstructive bronchitis.

34. The method of claim 33 wherein the respiratory tract disease is chronic bronchitis.

35. The method of claim 31 wherein the respiratory tract disease is chronic obstructive pulmonary disease.

36. The method of claim 31 wherein the respiratory tract disease is bronchial asthma.

37. The method of claim 31 wherein the respiratory tract disease is pneumonia.

38-55. (canceled)
